(19) United States
(12) Patent Application Publication
(10) Pub. No.: US 2005/0106671 A1
(43) Pub. Date: May 19, 2005

(54) Expression vector, host cell and method for producing fusion proteins

(76) Inventors: Darcy Birse, Quebec (CA); Anna-Karin Lundback, Sundbyberg (SE)

Correspondence Address: AMERSHAM BIOSCIENCES;PATENT DEPARTMENT, 800 CENTENNIAL AVENUE, PISCATAWAY, NJ, 08855, US

(21) Appl. No.: 10/990,718

(22) Filed: Nov. 17, 2004

(30) Foreign Application Priority Data
Nov. 18, 2003 (SE) 0303093-9

(51) Int. Cl. C12N 9/10; C12P 21/04; C07H 21/04

(52) U.S. Cl. 435/69.1; 435/193; 435/320.1; 435/325; 536/23.2

(57) ABSTRACT

The present invention relates to an expression vector comprising, in 5′ to 3′ direction, a promoter, a multiple cloning site and a nucleotide sequence encoding glutathione-S-transferase (GST), for production of a fusion protein comprising a membrane protein, secretory protein or toxic protein or peptide, fused directly or indirectly with the N-terminal of GST. Preferably, the fusion protein comprises GST and a membrane protein or membrane localised peptide. The invention is especially suitable for membrane proteins having their C-terminals in the cytoplasm. The invention also relates to methods for producing such fusion proteins using host cells transformed with the expression vector in which a desired gene has been cloned.

### FIELD OF THE INVENTION

The present invention relates to an expression vector, to a host cell transformed with said vector, and to methods of producing fusion proteins using said host cells. More closely, the invention relates to an expression vector for recombinant production of a fusion protein in which a membrane protein, secretory protein, toxic protein or peptide is fused with the enzyme glutathione-S-transferase (GST) at the N-terminal part of GST, i.e. the C-terminal part of the protein. Thus, the fusion protein will be expressed as a C-terminal tagged fusion protein.

### BACKGROUND OF THE INVENTION

The mechanisms behind the in vivo targeting and insertion of membrane proteins in the lipid bilayer are complex and not completely elucidated. Escherichia coli utilizes several targeting pathways for presecretory and integral membrane proteins. Proteins can either be co- or post-translationally targeted to the inner membrane of the cell. The polypeptides are then either inserted in the inner membrane, secreted in to the periplasm, or targeted to the outer membrane depending on the target sequence. For a fully active membrane protein, successful expression, targeting and insertion in to the lipid bilayer must be obtained.

For currently unknown reasons, a wide range of recombinant proteins, when expressed in a host cell strain (microbial, yeast, fungi or mammalian cell culture), cause a compromise in the function or health of the host cell, where this phenomenon is referred to as toxicity. Heterologous recombinant proteins, often expressed at relative high levels, can have a negative effect in the integrity of the host cell, are thereby identified as toxic proteins. To accommodate the expression of toxic proteins in a selected host cell strain, strategies can be applied to modify a given toxic protein (for example a fusion component) to aid in the tolerance of the toxic nature of the expressed protein, allowing for its expression with a moderate effect on the viability of the host cell strain.

Several vectors are known for recombinant production of foreign proteins some of which are designed to simplify the purification process of the desired protein.

The pGEX system described by Smith and Johnson utilises glutathione S-transferase as an N-terminal fusion partner [Smith, D. B. and Johnson, K. S. 1988. Single-step purification of polypeptides expressed in Escherichia coli as fusions with gluthatione S-transferase. Gene 67:31-40]. Many soluble but few membrane proteins have been expressed and purified using this system. The system provides an easy way to purify recombinant protein using glutathione immobilized on a chromatography gel. Fusion proteins containing GST-tags at the N-terminal of the protein are also described in U.S. Pat. No. 5,654,176 and applications in PCT/IB02/05740.

It has been shown that fusing of GST to the N-terminus of a target protein may decrease the activity and stability of the protein [Sharrocks, A. D. 1994. A T7 expression vector for producing N-and C-terminal proteins with gluthatione S-transferase. Gene. 138: 105-108]. It has been demonstrated that the activity of eukaryotic soluble DNA binding protein, RSRFCA, was unable to bind DNA if the protein was expressed with a N-terminal GST fusion. The DNA binding activity of the protein was not recovered after proteolytic cleavage of the GST tag. In the alternative experiment, when the protein was expressed with a C-terminal GST fusion, it remained active in terms of DNA binding activity.

Few recombinant membrane proteins have been expressed in heterologous expression systems with the desired activity and stability relative to native form. Furthermore, many recombinant membrane proteins with N-terminal fusion tags have been proven difficult to express in bacterial systems, such as E. coli. Therefore, there remains a great need for improvement within this technical field and its applications.

### SUMMARY OF THE INVENTION

An object of the present invention was to enable recombinant production of active and stable membrane proteins, secretory proteins, toxic proteins or peptides.

Another object of the present invention was to enable recombinant production of membrane proteins, secretory proteins, toxic proteins or peptides fused to GST.

A further object was to provide a suitable expression vector with a multiple cloning site for insertion of any desired membrane protein, secretory protein, toxic protein or peptide.

These and other objectives are fulfilled according to the invention. Thus, in a first aspect the invention provides an expression vector comprising, in 5′ to 3′ direction, a promoter, a multiple cloning site and a nucleotide sequence encoding glutathione-S-transferase (GST), for cloning of a nucleotide sequence encoding a membrane protein, secretory protein or toxic protein or peptide. This protein will, upon expression, be fused directly or indirectly with the N-terminal GST.

Thus, this aspect relates to use of an expression vector comprising, in 5′ to 3′ direction, a promoter, a multiple cloning site and a nucleotide sequence encoding glutathione-S-transferase (GST), for production of a fusion protein comprising a membrane protein, secretory protein or toxic protein or peptide, fused directly or indirectly with the N-terminal GST.

In a preferred embodiment, said fusion protein comprises GST and a membrane protein, or membrane localised peptide, secretory protein, toxic protein or peptide. The membrane protein, secretory protein, toxic protein or peptide may be with or without signal sequence or signal peptide. However, the invention also concerns other proteins/peptides having signal sequences, such as secretory proteins/peptides with N-terminal signal sequences.

The invention is especially useful for recombinant production of membrane proteins having their C-terminal in the cytoplasm.

The invention is also specifically useful for recombinant production of toxic proteins that require a neutralizing toxicity effect observed through having a GST moiety fused at the C-terminal of the target protein, tolerating toxic protein expression in a given cell strain.

In the expression vector, the membrane protein, secretory protein, toxic lo protein or peptide is preferably fused to GST through a cleavable linker sequence, such as by a by a site specific protease, preferably PreScission protease™.

A preferred promoter for expression of the fusion proteins is tac. Alternative promoters are CMV, RSV, SV40, etc. depending on the selected host cell.

According to a preferred embodiment of the invention the multiple cloning site comprises at least two, preferably all, of the following restriction endonuclease recognition sites: HindII; BamHI; EcoRI; SmaI; SaII; XhoI, which are localized between the promoter and the GST fusion tag. This multiple cloning site enables cloning of a wide range of nucleic acids and is especially suitable for recombinant production of membrane proteins or peptides.

Preferably, the expression vector comprises a PGEX plasmid.

The most preferred expression vector according to the invention comprises the nucleotide sequence according to SEQ. ID NO 11 in the Sequence Listing.

According to a second aspect of the invention, there is provided a host cell comprising, or transformed with, the expression vector described above, wherein a nucleotide sequence encoding a membrane protein, secretory protein or toxic protein or peptide has been cloned into a desired site in the multiple cloning site region. Preferably, the host cell is a bacterial cell, such as E. coli, but can also be any type of microbial, yeast, fungi or mammalian cell strain. In any case, suitable promoters and selection markers are chosen according to the selected host cell and conventional practice.

According to a third aspect of the invention, there is provided a method of producing a fusion protein comprising GST and a membrane protein, secretory protein, toxic protein or peptide or signal-sequence-containing protein/peptide fused directly or indirectly with the N-terminal of GST, which method comprises:
a) transforming a host cell, for example E. coli, with an expression vector as above and having therein inserted a nucleic acid sequence encoding a membrane protein, secretory protein or toxic protein or peptide;
b) culturing said host cell under conditions such that said fusion protein is expressed in recoverable quantities,
c) recovering fusion proteins from said host cells,
d) purifying said fusion proteins.

In a preferred embodiment, the method comprises a detergent screening procedure (Life Science News, October 2003 issue) to assay for GST activity of the GST-fusion target membrane protein, secretory protein, toxic protein or peptide.

Preferably, step d) is by affinity purification, preferably affinity chromatography using gluthatione or an antibody directed against GST (or antigen binding fragment thereof) as a ligand.

In a further embodiment, the method comprises the following steps:
e) cleaving said membrane protein or secretory protein or toxic protein or peptide from said GST; and
f) isolating said membrane protein or secretory protein or toxic protein or peptide.

In an especially advantageous embodiment of the method, step e) is performed by on-column cleavage. Preferably, this is enabled by the use of PreScission protease™.

The method is expected to be especially useful for production of membrane proteins. Preferably, membrane proteins having their C-terminal in the cytoplasm. The invention is contemplated to be very useful in the production of mammalian membrane proteins as drug targets for use in high throughput screening of new drugs acting on membrane proteins.

According to the present invention, the GST tag is fused to the C-terminal of the membrane protein, to avoid any interference with the target sequence. In this way, the large GST tag does not have to be translocated (FIG. 1). In most organisms, about 60 to 70% of the multi-spanning α-helical membrane proteins are predicted to have their C-terminal located in the cytoplasm. According to the present invention a GST-tag is fused to the C-terminal of these membrane proteins which gives a higher expression yield than if the GST tag was fused to the N-terminal.

The present invention provides an expression vector (FIG. 2) for cloning of recombinant membrane proteins or peptides with a C-terminal GST fusion. The vector could be used not only for membrane proteins but also for any protein comprising a signal sequence, for example secretory proteins or peptides.

### BRIEF DESCRIPTION OF THE DRAWINGS

FIG. 1 is a schematic view showing an expressed membrane protein with C-terminal GST-tag.

FIG. 2 is a schematic view of an expression vector according to the invention for production of recombinant proteins with a C-terminal GST-fusion (SEQ ID NO 12).

FIG. 3 shows primers to amplify the reversed GST casette. The sequences for the restriction enzyme sites used for cloning are in bold letters (SEQ ID NO 9 and SEQ ID NO 10).

FIG. 4 shows the strategy for producing the vector of the invention: a) create a reversed GST cassette from the pGEX-2T vector with engineered primers (also containing HindIII and NotI sites for cloning); b) introduce the HindIII site in the pGEX-6p-1 vector; c) remove the GST cassette by digesting the plasmid with HindIII and NotI; d) ligate a) and c).

FIG. 5 shows the results of a restriction analysis of pGEX-6p-1 vector on a 1.0% agarose gel stained with ethidium bromide.

FIG. 6 shows the results of a restriction analysis of the HindIII mutated vector on a 1.0% agarose gel stained with ethidium bromide.

FIG. 7 shows a restriction digest of the vector of the invention compared to the Hindlll mutated pGEX-6p-1 vector to evaluate of the ligation was successful.

FIG. 8 shows the sequence of the C-terminal vector of the invention also shown in the sequence listing as SEQ ID NO 11.

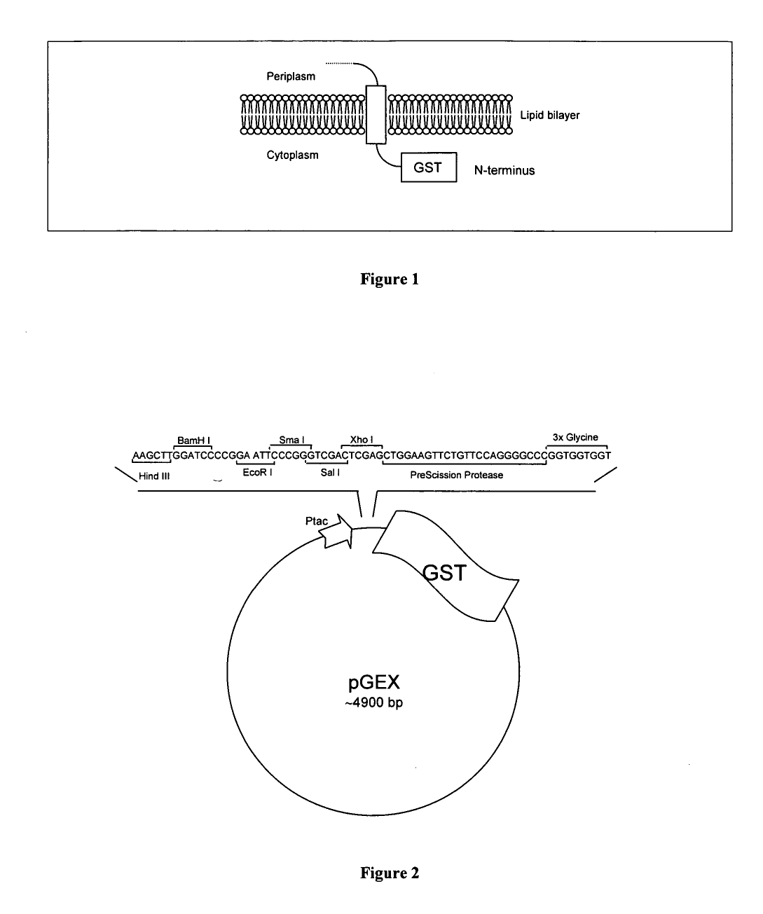

### DETAILED DESCRIPTION OF THE INVENTION

Materials and Methods

All restriction enzymes utilized in the experiments listed herein were from Amersham Biosciences unless otherwise stated. pGEX-vectors were from Amersham Biosciences and QuickChange Site-Directed Mutagenesis kit from Stratagene. QIAquick PCR Purification Kit and QIAprep® Miniprep Kit was purchased from QIAGEN. T4 DNA ligase and Taq DNA polymerase were from GIBCO unless otherwise stated. TOPO-TA cloning kit was purchased from Invitrogen and Pwo DNA polymerase was from Roche.

### EXAMPLES

Below, the present invention will be illustrated by way of examples. However, the present examples are provided for illustrative purposes only and should not be construed as limiting the present invention as defined by the appended claims. All references given below and elsewhere in the present specification are hereby included herein by reference.

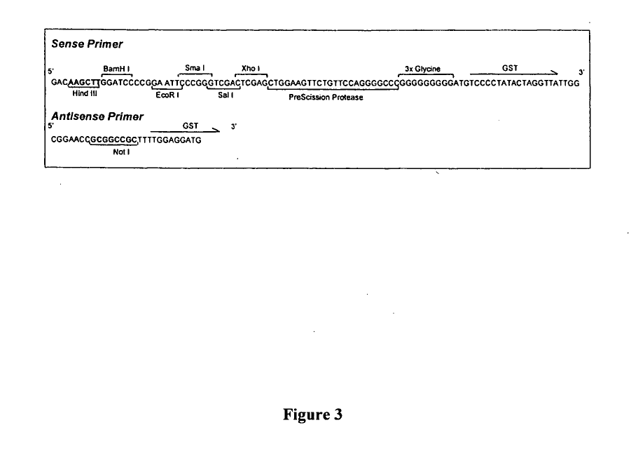

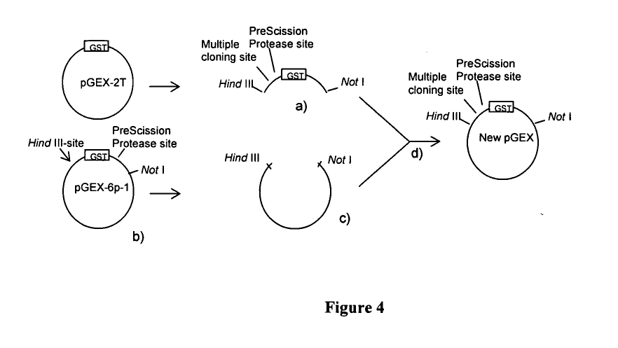

Two commercially available GST-vectors (pGEX-2T and pGEX-6p-1) were used as starting points for construction of the new C-terminal GST fusion vector. The strategy was to construct a DNA fragment where the multiple cloning site and the PreScission Protease site were situated upstream the GST gene (the reversed GST cassette) and then clone the fragment into the pGEX-6p-1 vector. To accomplish this: a) the reversed GST cassette was amplified from the pGEX-2T vector with primers. The sense primer (SEQ ID NO 9) contained the sequence for the multiple cloning site, PreScission Protease site and also a Hind III site for cloning (FIG. 3). The antisense primer (SEQ ID NO 10) contained a Not I site for cloning. The pGEX-2T was used to eliminate any false primer binding, since it does not contain a Precission Protease site. b) a Hind III site was introduced downstream the Tac promotor in the pGEX-6p-1 vector for cloning and to remove the existing GST gene, the PreScission Protease site and multiple cloning site (the GST cassette). c) the GST cassette was removed by digesting the plasmid with the restriction enzymes Hind III and Not I. The Not I site was the last restriction site in the multiple cloning site. d) the digested pGEX-6p-1 vector and the reversed GST cassette was ligated to create the new C-terminal GST vector (FIG. 4). pGEX-6p-1 vector

Restriction Analysis

To ensure that the vector did not already contain a Hind III restriction site, the plasmid was digested with Hind III. As a positive control the plasmid was also digested with BamH I and as a negative control the plasmid was digested with Nae I, which is not supposed to cleave the vector. 2 μL of pGEX-6p-1 plasmid was incubated with 15 U of Hind III, 15 U of BamH I and 10 U of Nae I for three hours at 37° C. The effect of the salt concentration in the different buffers on the migration of DNA in an agarose gel was also investigated. The plasmid was mixed with the three different buffers K (10 mM MgCl₂ and 100 mM NaCl), L (10 mM MgCl₂) and M (10 mM MgCl₂ and 50 mM NaCl). The samples were analysed by agarose gel electrophoresis.

Mutagenesis

A Hind III restriction site was introduced in the pGEX-6p-1 vector with the QuickChange Site-Directed Mutagenesis kit according to the manufacturer's protocol. The PfuTurbo® DNA polymerase of the kit transcribes both of the plasmid strands simultaneously and introduces the mutation with two complementary primers. After the mutation is completed the parental DNA template is digested with Dnp I, an endonuclease which is specific for methylated and hemimethylated DNA. The new plasmid is nicked but will be repaired upon transformation into E. coli. The sequence of the sense primer was: 5′ C ATG TCC CCT AAG CTT GGT TAT TGG AM ATT MG GG 3′ (SEQ ID NO 1) and the sequence of the antisense primer was: 5′ CCC TTA ATT TTC CM TM CCA AGC TTA GGGT GAC ATG MT AC 3′ (SEQ ID NO 2) (the mutation site is underlined). The PCR was carried out in a PCR System 9700 (Gene Amp).

Transformation and Plasmid Isolation

The Hind III mutated plasmid was transformed into Ca²⁺ competent MC1061 cells by heat shocking. An aliquot of 1 μL plasmid was added to 50 μL of Ca²⁺ competent E. coli XL1 Blue cells and incubated on ice for 30 minutes. The cells were heat-shocked at 42° C. for 45 seconds and put directly on ice for two minutes. They were then suspended in 500 μL of SOC (2% tryptone, 0.5% yeast extract, 10 mM NaCl, 2.5 mM KCl, 10 mM MgCl₂, 10 mM MgSO₄ and 20 mM glucose) and incubated in a floor shaker at 220 rpm at 37° C. to grow for one hour. The cells were spread on a LA plate supplemented with 50 μg/mL carbenicillin and incubated over night at 37° C. Colonies from the transformation plate were selected and inoculated in 5 ml LB supplemented with 100 μg/mL carbenicillin. Cells were incubated over night in a floor shaker 220 rpm at 37° C. The mutated plasmid was isolated and purified using QIAprep® Miniprep Kit according to the manufacturer's protocol. The plasmid was eluted in 50 μL sterile water.

Restriction Analysis

A restriction analysis was made on the plasmid to confirm that the mutation was successful. The plasmid (3 μL) was incubated at 37° C. for three hours with 15 U of Hind III. A double digested was also done by adding 15 U of Hind III and 15 U of BamH I to 3 μL plasmid. The samples were separated by agarose electrophoresis.

Reversed GST Cassette

Restriction Digest

The reversed GST cassette was amplified by PCR from the pGEX-2Tvector with engineered primers. The vector did already contain a gene for a protein. To eliminate any false priming the vector was first digested with EcoR V and BamH I (enzymes from Gibco). An aliquot of 20 μL of plasmid was digested with 20 U of EcoR V and 20 U of BamH I at 37° C. for 3 hours. The sample was analysed by agarose electrophoresis and a fragment (1800 bp) was excised from the gel. The DNA fragment was purified using QIAquick PCR Purification Kit according to the manufacturer's protocol. The concentration was estimated by measuring the absorbance at 260 nm on a spectrophotometer (UV-1601 Shimadzu).

PCR

The purified DNA fragment from above was used to produce the reversed GST cassette. An aliquot of DNA (100 ng) was mixed with 3.5 U of Pwo DNA polymerase and 125 ng of each primer (FIG. 3). The PCR was performed on a Gene Amp® PCR System 9700 with a lid preheated to 103° C. The samples were first denatured at 94° C. for 2 minutes. 25 cycles where then repeated as follows: 15 seconds at 94° C., 30 seconds at 50° C. and 30 seconds at 72° C. The PCR product was analysed by agarose gel electrophoresis and purified with the QIAquick PCR Purification Kit according to the manufacturer's protocol. The product was eluted in 50 μL sterile water.

Ligation

The Hind III mutated vector and the reversed GST cassette were digested with the restriction enzymes Not I and Hind III (enzymes from Gibco). An aliquot of 20 μL plasmid was digested for 3 hours at 37° C. with 20 U of Not 1. Hind III (20 U) was added and the samples were incubated a further 3 hours at 37° C. The products were separated by low melting agarose gel electrophoresis and the gel pieces containing the fragments of right size (˜700 bp and ˜4200 bp) were excised from the gel. The gel pieces were melted at 70° C. and 5 μL of vector and 10 μL insert were mixed with 1 U of T4 DNA ligase. The samples were incubated at 16° C. for 20 hours. The ligation mix (20 μL) was melted at 70° C. and mixed with 20 μL of 2xTCM buffer (20 mM Tris-HCl, 20 mM CaCl₂ and 20 mM MgCl₂, pH 7.5). An aliquot of 100 μL of Ca²⁺ competent E. coli MC1061 cells was added to the mixture and incubated on ice for one hour. The cells were heat-shocked at 42° C. for 45 seconds and put directly on ice for two minutes. An aliquot of 800 μL of SOC was added and the suspension was incubated in a floor shaker at 37° C. to grow for one hour. Cells were spread on a LA plate containing 50 μg/mL carbenicillin and incubated over night at 37° C.

TOPO-TA Cloning

Several attempts to ligate the reversed GST cassette with the Hind III mutated pGEX-6p-1 vector failed. Various amounts of DNA and digestion times were tested without success. One possible explanation for this may be that the Not I site in the GST-fragment was too close to the 3′ end. The Not I enzyme requires a large overhanging sequence to efficiently digest the DNA. To resolve this problem, the fragment was cloned into a vector prior to cleavage with restriction enzymes. The TOPO-TA cloning system allows for the PCR fragment to be cloned into the vector if the DNA has a single deoxyadeonosine overhang. The Taq polymerase always produces an adenosine overhang but lacks proofreading activity resulting in the possible introduction of mismatched bases approximately every 1000 bp.

PCR and Cloning

A mixture of 100 ng of DNA template, 125 ng of each primer, 5 μL of 10× reaction buffer (200 mM Tris-HCl, 500 mM KCl, pH 8.4) and 5 U Taq DNA polymerase was prepared. PCR was done for 25 repeated cycles as follows: 15 seconds at 94° C., 30 seconds at 50° C. and 30 seconds at 72° C. on a Gene Amp® PCR System 9700 with a lid preheated to 103° C. The last elongation step was prolonged to 10 minutes to ensure that the Taq DNA polymerase added the adenoside overhang. The PCR product was directly cloned into the pCRII®-TOPO® vector (Invitrogen) according to the manufacturer's protocol. The clones were transformed into TOP10F′® cells (Invitrogen) according to the manufacturer's protocol and spread on a LA plate containing 50 μg/mL carbenicillin and 40 μL of 40 mg/mL X-gal, 40 μL of 100 mM IPTG (for white/blue screening). The plates were incubated at 37° C. over night. Plasmids were isolated and purified with QIAprep® Miniprep Kit and eluted with 50 μL sterile water.

Restriction Digest, Ligation and Plasmid Isolation

The pCR®II-TOPO® vector containing the PCR fragment and the Hind III mutated pGEX-6p-1 vector were both digested with the restriction enzymes Not I and Hind III, by adding 64 U of Not I to 5 μL plasmid. The two samples were digested overnight at 37° C. to ensure that the digestion was complete. Since Hind III has been observed to have non-specific cleavage activity during prolonged DNA digestion reactions, the plasmids were digested subsequently by Not I digestion followed by digestion with 60 U of Hind III for 4 hours at 37° C. The digested plasmids were separated on 1% low melting agarose gel and the desired DNA fragments were excised from the gel. The DNA was then ligated and transformed into the MC1061 cell line. Plasmids were isolated with QIAprep® Miniprep Kit according to the manufacturer's protocol and eluted in 50 μL sterile water.

Restriction Analysis

To screen for success full clones, 5 μl plasmid and 5 μL of the Hind III mutated pGEX-6p-1 plasmid were separately double digested with 2 U Bal I+76 U Not I and with 2 U Bal I+30 U Hind III 37° C. for 4 hours. Digested successful clones would produce bands with shifts compared to the Hind III mutated pGEX-6p-1 vector. The digested samples were separated by agarose gel electrophoresis.

Sequencing and Correction

The plasmid, shown by agarose gel electrophoresis to have a correct restriction pattern was sent for sequence analysis at Cybergene, Huddinge. The sense primer for sequencing was: 5′ GCT GTT GAC MT TM TCA TCG GC 3′ (SEQ ID NO 3) and the antisense primer was: 5′ GCA TGT GTC AGA GGT TTT CAC CG 3′ (SEQ ID NO 4). Both primers were synthesised by Cybergene. The sequencing showed a deletion and a mis-match, which were corrected by another round of mutagenesis experiments with primers that corrected (deleted) the errors. The mutation was carried out using the QuickChange Mutagenesis kit according to the manufacturer's protocol. The sequence of the sense primer was: 5′ CCC GGG GGG GGG ATG TCC CCT ATA CTA GGT TAT TGG 3′ (SEQ ID NO 5) and the sequence of the antisense primer was: 5′ CCA ATA ACC TAG TAT AGG GGA CAT CCC CCC CCC GGG 3′ (SEQ ID NO 6). Both primers synthesised by Cybergene, Huddinge. The letters in bold Italics correspond to the inserted nucleotides and the bold underlined letters correspond to the changed nucleotide. Plasmids were isolated and sent for final sequencing (Cybergene, Huddinge).

Second Correction and Sequencing

Following interpretation of the sequencing results, there was a construction error in the vector, which produced an unwanted Sma I site. This error was corrected by a second mutagenesis reaction with the QuickChange Mutagenesis kit (Stratagene) using new primers. The codon for glycine was changed from GGG to GGT. The sequence for the sense primer for mutagenesis was: 5′ CGA GGG GCC CGG TGG TGG TAT GTC C CCT ATA C 3′ (SEQ ID NO 7) and the sequence for the antisense primer was: 5′ GTA TAG GGG ACA TAC CAC CAC CGG GCC CCT 3′ (SEQ ID NO 8) (synthesised by Cybergene, Huddinge). The bold letters correspond to the changed nucleotides. Plasmids were isolated and sent for a third sequence analysis (Cybergene, Huddinge).

Agarose Gel

Samples were mixed with 6× loading buffer (0.25% bromophenol blue, 0.25% xylene cyanol FF, 30% glycerol in water) and loaded on a 1% agarose gel supplemented with ethidium bromide. The samples were separated at 120 V for 45 minutes with TAE buffer (40 mM Tris-acetate, 1 mM EDTA) as running buffer.

Results

Restriction Analysis

Hind III Mutation

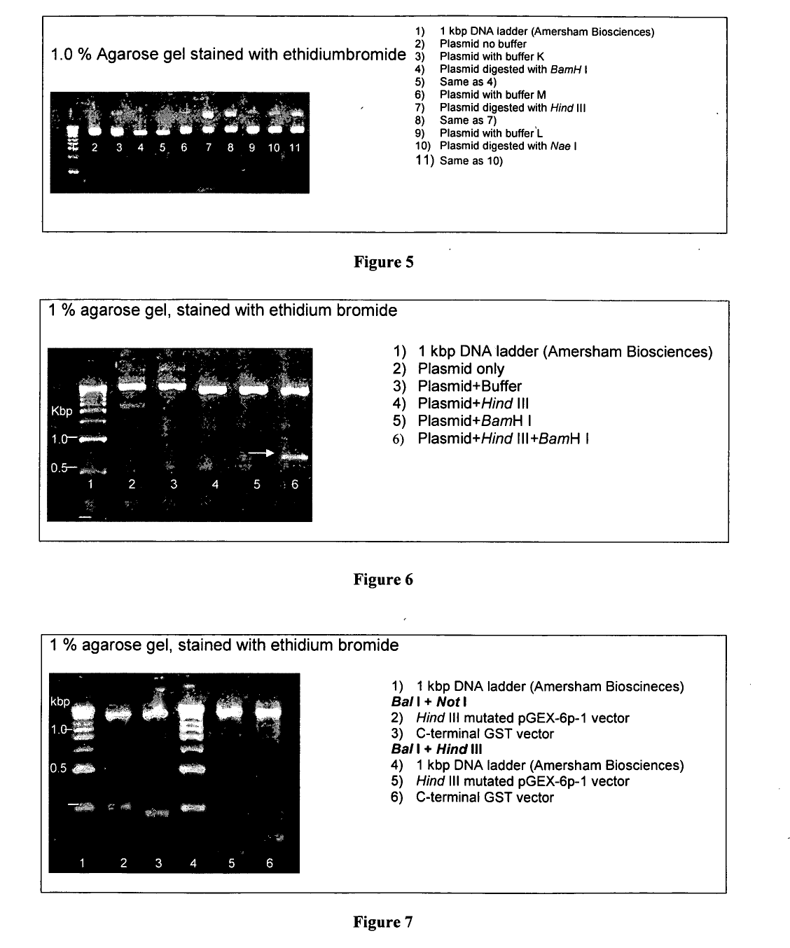

The restriction analysis of the pGEX-6p-1 vector before the mutation with Hind III showed that there was no Hind III site in the vector. The plasmid was not linearised with Hind III or Nae I, but with BamH I as expected. The plasmid was also tested with three different buffers K, L and M to see how the salt concentration effects the migration of the DNA. The buffers used gave essentially the same effect on the DNA migration (FIG. 5). A single cleaved plasmid migrates faster than an untreated plasmid. Restriction analysis showed that the mutation was successful. The Hind III mutated pGEX-6p-1 vector double digested with Hind III and BamH I produced, as expected, a 680 bp fragment (FIG. 6).

Ligation

To evaluate whether the ligation of the reversed GST cassette and the Hind III mutated pGEX-6p-1 vector had been successful, the new vector was separately double digested with Bal I +Not I and Bal I+Hind III. The new vector digested with Bal I+Not I would produce a 59 bp smaller fragment compared to the Hind III mutated pGEX-6p-1 vector. The new vector digested with Bal I+Hind III would produce a 74 bp larger fragment compared to the Hind III mutated pGEX-6p-1 vector if the ligation was successful. As judging from the agarose gel electrophoresis analysis the ligation was successful (FIG. 7).

Sequencing

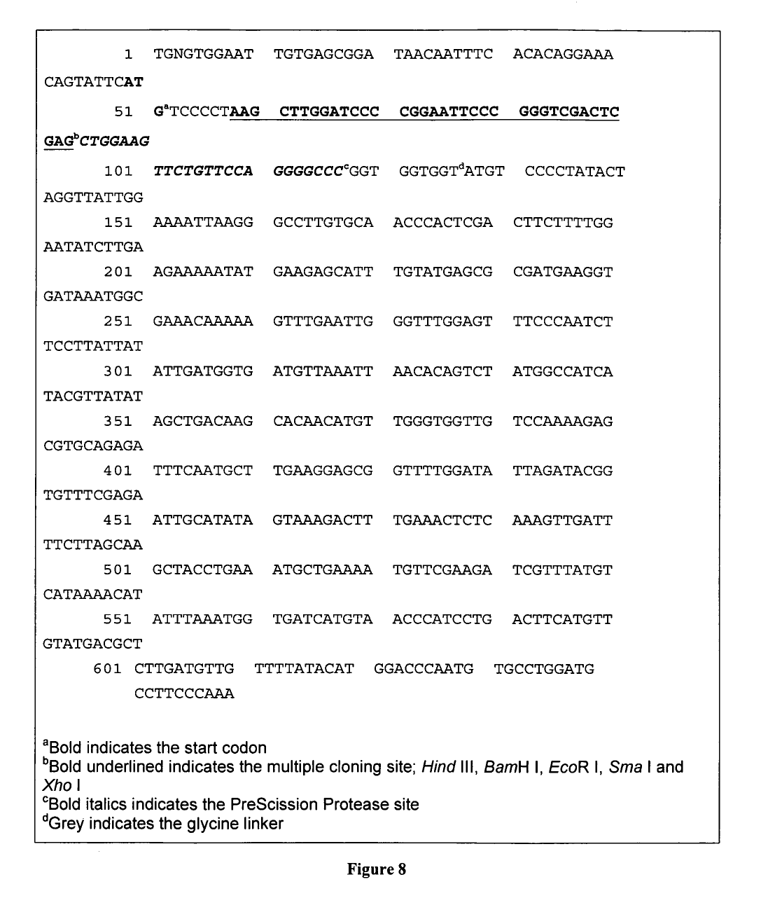

The third sequencing proved that the second mutagenesis as well as the first was successful and the C-terminal GST vector had the desired sequence (FIG. 8) (SEQ ID NO 11).

It is apparent that many modifications and variations of the invention as hereinabove set forth may be made without departing from the spirit and scope thereof. The specific embodiments described are given by way of example only, and the invention is limited only by the terms of the appended claims.

1. An expression vector comprising, in 5′ to 3′ direction, a promoter, a multiple cloning site and a nucleotide sequence encoding glutathione-S-transferase (GST) having an N-terminal, for cloning of a nucleotide sequence, wherein said nucleotide sequence, upon expression, is fused directly or indirectly with the N-terminal of GST.

2. The expression vector of claim 1, wherein the multiple cloning site includes the restriction endonuclease recognition sites HindIII, BamHI, EcoRI, SmaI, Sal I, XhoI, which are localised between the promoter and the GST tag.

3. The expression vector of claim 1, further comprising a cleavable link situated between the multiple cloning site and the nucleotide sequence encoding GST.

4. The expression vector of claim 3, wherein said cleavable link is cleavable by a site specific protease.

5. The expression vector of claim 4, wherein said site specific protease is PreScission protease™.

6. The expression vector of claim 1, wherein the promoter is tac.

7. The expression vector of claim 1, which comprises a pGEX plasmid.

8. The expression vector of claim 1, comprising the nucleotide sequence of-SEQ. ID NO 11.

9. A host cell comprising the expression vector of claim 1, further comprising a nucleotide sequence encoding a membrane protein, secretory protein or toxic protein/peptide cloned into a site in the multiple cloning site.

10. The host cell of claim 9, which is a bacterial, yeast, fungi or mammalian cell.

11. The host cell of claim 10, which is E. coli.

12. A method of producing a fusion protein including GST and a membrane protein, secretory protein or toxic protein/peptide, fused directly or indirectly with the N-terminal of GST, which method comprises:
a) culturing a host cell under conditions such that said fusion protein is expressed in recoverable quantities,
b) recovering fusion proteins from said host cells, and
c) purifying said fusion proteins.

13. The method of claim 12, further comprising, between step b) and c), a detergent screening of said fusion purified fusion proteins to assay for GST activity of the GST-fusion target membrane protein, secretory protein or toxic protein or peptide.

14. The method of claim 12, wherein the purifying step d) is by affinity chromatography.

15. The method of claim 12, further comprising the steps:
d) cleaving said protein/peptide from said GST; and
e) isolating said protein/peptide.

16. The method of claim 15, wherein the cleaving step is performed by on-column cleavage.

17. The method of claim 12, wherein the vector is as defined in SEQ ID NO 11 and the host cell is E.coli.


SEQUENCE LISTING (12 sequences)

SEQ ID NO: 1 - DNA, 36 bp, artificial sequence
  synthetic oligonucleotide
catgtcccctaagcttggttattggaaaattaaggg

SEQ ID NO: 2 - DNA, 42 bp, artificial sequence
  synthetic oligonucleotide
cccttaattttccaataaccaagcttagggtgacatgaatac

SEQ ID NO: 3 - DNA, 23 bp, artificial sequence
  synthetic oligonucleotide
gctgttgacaattaatcatcggc

SEQ ID NO: 4 - DNA, 23 bp, artificial sequence
  synthetic oligonucleotide
gcatgtgtcagaggttttcaccg

SEQ ID NO: 5 - DNA, 36 bp, artificial sequence
  synthetic oligonucleotide
cccgggggggggatgtcccctatactaggttattgg

SEQ ID NO: 6 - DNA, 36 bp, artificial sequence
  synthetic oligonucleotide
ccaataacctagtataggggacatcccccccccggg

SEQ ID NO: 7 - DNA, 32 bp, artificial sequence
  synthetic oligonucleotide
cgaggggcccggtggtggtatgtcccctatac

SEQ ID NO: 8 - DNA, 30 bp, artificial sequence
  synthetic oligonucleotide
gtataggggacataccaccaccgggcccct

SEQ ID NO: 9 - DNA, 96 bp, artificial sequence
  synthetic oligonucleotide
gacaagcttggatccccggaattcccgggtcgactcgagctggaagttctgttccaggggcccgggggggggatgtcccctatactaggttattgg

SEQ ID NO: 10 - DNA, 27 bp, artificial sequence
  synthetic oligonucleotide
cggaaccgcggccgcttttggaggatg

SEQ ID NO: 11 - DNA, 650 bp, artificial sequence
  synthetic oligonucleotide
tgngtggaattgtgagcggataacaatttcacacaggaaacagtattcatgtcccctaagcttggatccccggaattcccgggtcgactcgagctggaagttctgttccaggggcccggtggtggtatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgccttcccaaa

SEQ ID NO: 12 - DNA, 69 bp, artificial sequence
  synthetic oligonucleotide
aagcttggatccccggaattcccgggtcgactcgagctggaagttctgttccaggggcccggtggtggt